(19) United States
(12) Patent Application Publication
(10) Pub. No.: US 2025/0110032 A1
(43) Pub. Date: Apr. 3, 2025

(54) SYSTEMS AND METHODS FOR MANUFACTURING AND EVALUATING PERFORMANCE OF CONCRETE PRODUCTS

(71) Applicant: CARBICRETE INC., Montreal (CA)

(72) Inventors: Mehrdad MAHOUTIAN, Montréal (CA); Chris STERN, Westmount (CA); Martin LATOUR, Montreal (CA)

(21) Appl. No.: 18/899,520

(22) Filed: Sep. 27, 2024

Related U.S. Application Data
(60) Provisional application No. 63/586,030, filed on Sep. 28, 2023.

(51) Int. Cl. G01N 3/30; B65B 57/00; C04B 40/00; G01N 29/07; G01N 33/38

(52) U.S. Cl. CPC G01N 3/30; B65B 57/00; C04B 40/0096; G01N 29/07; G01N 33/383

(57) ABSTRACT

A method for detecting non-compliance with performance requirements of concrete products includes receiving the concrete products from a curing chamber after curing of the concrete products, and, as the concrete products are displaced from the curing chamber to a packaging station, non-destructively testing the cured concrete products for compliance with performance requirements. A non-compliant concrete product that fails to meet one or more of the performance requirements is identified and removed before the non-compliant concrete product is packaged in the packaging station, wherein the non-compliant concrete product has a performance indicator below a threshold.

### CROSS-REFERENCES TO RELATED APPLICATIONS

This application claims benefit from United Stated Patent Application No. 63/586,030 filed on Sep. 28, 2023, the entire content of which is incorporated herein by reference.

### TECHNICAL FIELD

The application relates generally to concrete products and, more particularly, to systems and methods used to evaluate mechanical properties of such concrete products.

### BACKGROUND

When manufacturing precast concrete products, the products are packaged and shipped to construction sites with little or no quality control. In the case of concrete masonry units, the units are removed from the curing room, visually inspected, and packaged. Neither destructive or non-destructive testing is performed on the blocks before they are sent out of the concrete plant. Returning the blocks through a test cycle may increase manipulation and associated health and safety risks, potential damages to the blocks, and/or increase the overall production turnover time and costs.

### SUMMARY

In one aspect, there is provided a method for manufacturing a concrete product, comprising: performing, in a curing chamber, a carbonation curing process on a formed intermediate of the concrete product; after the carbonation curing process, displacing the concrete product from the curing chamber to a packaging station; and during the displacing of the concrete product from the curing chamber to the packaging station, non-destructively testing performance of the concrete product.

The method as defined above and described herein may include any one or more of the features, in whole or in part, and in any combination.

In some embodiments, the non-destructively testing the performance of the concrete product includes moving the concrete product through an evaluation station containing non-destructive testing equipment.

In some embodiments, the non-destructive testing equipment is supported by a fixture, the non-destructive testing equipment being movable relative to the fixture, the method comprising moving the non-destructive testing equipment towards the concrete product.

In some embodiments, the non-destructive testing equipment is supported by a fixture, the non-destructive testing equipment being non-movable relative to the fixture, the method comprising moving the concrete product towards the non-destructive testing equipment.

In some embodiments, the non-destructive equipment includes one or more of a rebound hammer and an ultrasonic pulse velocity device.

In some embodiments, the non-destructively testing of the performance of the concrete product includes evaluating a compressive strength of the concrete product.

In some embodiments, the method includes striking the concrete product with a rebound hammer.

In some embodiments, the non-destructively testing of the performance of the concrete product includes evaluating a continuity of the concrete product.

In some embodiments, the method includes subjecting the concrete product to a pulse generated by an ultrasonic pulse velocity device.

In some embodiments, the displacing of the concrete product includes displacing the concrete product via a conveyor belt.

In some embodiments, the method includes weighting the concrete product while the concrete product is on the conveyor belt.

In another aspect, there is provided a method for detecting non-compliance with performance requirements of concrete products, comprising: receiving the concrete products from a curing chamber after curing of the concrete products; as the concrete products are displaced from the curing chamber to a packaging station, non-destructively testing the concrete products for compliance with performance requirements; and identifying a non-compliant concrete product that fails to meet one or more of the performance requirements, and removing the non-compliant concrete product before the non-compliant concrete product is packaged in the packaging station, the non-compliant concrete product having a performance indicator below a threshold.

The method as defined above and described herein may include any one or more of the features, in whole or in part, and in any combination.

In some embodiments, the non-destructively testing the performance of the concrete product includes moving the concrete product through an evaluation station containing non-destructive testing equipment.

In some embodiments, the non-destructive testing equipment is supported by a fixture, the non-destructive testing equipment being movable relative to the fixture, the method comprising moving the non-destructive testing equipment towards the concrete product.

In some embodiments, the non-destructive equipment includes one or more of a rebound hammer and an ultrasonic pulse velocity device.

In some embodiments, the non-destructively testing of the performance of the concrete product includes evaluating a compressive strength of the concrete product.

In some embodiments, the method includes striking the concrete product with a rebound hammer.

In some embodiments, the non-destructively testing of the performance of the concrete product includes evaluating a continuity of the concrete product.

In some embodiments, the method includes subjecting the concrete product to a pulse generated by an ultrasonic pulse velocity device.

In some embodiments, the displacing of the concrete product includes displacing the concrete product via a conveyor belt.

In some embodiments, the method includes weighting the concrete product while the concrete product is on the conveyor belt.

In some embodiments, the non-destructively testing of the performance of the concrete products includes simultaneously testing performance of a plurality of the concrete products.

In some embodiments, the simultaneously testing of the performance of the plurality of the concrete products includes moving a plurality of non-destructive testing devices relative to the plurality of the concrete products and non-destructively testing each of the plurality of the concrete products using a respective one of the non-destructing testing devices.

In yet another aspect, there is provided a method of manufacturing concrete products, comprising: obtaining concrete products via a process including: mixing a composition including a binder, an aggregate, and water to produce a concrete mixture, imparting a form to the concrete mixture to provide a formed intermediate, conditioning the formed intermediate to obtain a conditioned intermediate, and curing the conditioned intermediate with a gas containing carbon dioxide to obtain the concrete product; non-destructively testing a subset of the concrete products for compliance with performance requirements; determining that the subset of the concrete products fails to meet one or more of the performance requirements; and modifying one or more of the composition and the process by changing one or more of a binder content of the binder, an aggregate content of the aggregate, a curing time, a concentration of the carbon dioxide in the gas, a concentration of the carbon dioxide in a curing chamber in which the conditioned intermediate is cured, a pressure of the gas, and a water content of the water until the concrete products meet the performance requirements.

The method as defined above and described herein may include any one or more of the features, in whole or in part, and in any combination.

In some embodiments, the non-destructively testing of the concrete product includes moving the concrete product through an evaluation station containing non-destructive testing equipment.

In some embodiments, the non-destructive testing equipment is supported by a fixture, the non-destructive testing equipment being movable relative to the fixture, the method comprising moving the non-destructive testing equipment towards the concrete product.

In some embodiments, the non-destructive equipment includes one or more of a rebound hammer and an ultrasonic pulse velocity device.

In some embodiments, the non-destructively testing of the performance of the concrete product includes evaluating a compressive strength of the concrete product.

In some embodiments, the method includes striking the concrete product with a rebound hammer.

In some embodiments, the non-destructively testing of the performance of the concrete product includes evaluating a continuity of the concrete product.

In some embodiments, the method includes subjecting the concrete product to a pulse generated by an ultrasonic pulse velocity device.

In some embodiments, the method includes displacing of the concrete product includes displacing the concrete product via a conveyor belt.

In some embodiments, the modifying of the composition includes one or more of: reducing the binder content by 1% by weight if an average compressive strength of the subset of the concrete products is at least 5% higher than a minimum compressive strength requirement; reducing a curing time of the curing of the conditioned intermediate by at least 5% if the average compressive strength of subset of the concrete products is at least 5% higher than the minimum compressive strength requirement; reducing a carbon dioxide concentration of the gas containing carbon dioxide by at least 5% if the average compressive strength of subset of the concrete products is at least 5% higher than the minimum compressive strength requirement; reducing a pressure of the gas containing carbon dioxide by at least 5% if the average compressive strength of subset of the concrete products is at least 5% higher than the minimum compressive strength requirement.

In some embodiments, the non-destructively testing of the performance of the concrete products includes simultaneously testing performance of a plurality of the concrete products by moving a plurality of non-destructive testing devices relative to the plurality of the concrete products and non-destructively testing each of the plurality of the concrete products using a respective one of the non-destructing testing devices.

### BRIEF DESCRIPTION OF THE DRAWINGS

Reference is now made to the accompanying figures in which:

FIG. 1 is a schematic view of a system used for curing and conditioning a concrete product;

FIG. 2 is a schematic view of a system for manufacturing, evaluating performances, and packaging concrete products in accordance with one embodiment;

FIG. 3 is a flowchart illustrating steps of a method of manufacturing a concrete product in accordance with one embodiment;

FIG. 4 is a flowchart illustrating steps of a method of detecting non-compliance with performance requirements of concrete products;

FIG. 5 is a flowchart illustrating steps of a method of manufacturing concrete products in accordance with another embodiment; and

FIG. 6 is a schematic representation of a controller in accordance with one embodiment.

### DETAILED DESCRIPTION

### Introduction

There remains growing interest worldwide to reduce the environmental footprint of precast concrete. Carbonation curing technology is among the most promising solutions. During carbonation curing, precast concrete hardens mainly through a so-called carbonation reaction which happens between carbon dioxide and the oxides, and/or hydroxide of calcium and/or magnesium, with the existence of water. Under appropriate raw material selection, mix design and process control, carbonated precast concrete may be as strong and durable as traditional precast concrete, and suitable for a variety of applications.

Manufacturing precast concrete with carbonation curing technology or mineralization may address concerns over climate change. Under appropriate processing condition, freshly cast concrete products may achieve rapid hardening when being exposed in CO₂-rich environment. This CO₂ sequestration may help to mitigate the CO₂ emissions associated with the construction industry. Other advantages of carbonated precast concrete may include the improvement of productivity through rapid hardening, the reduction of production costs through the replacement of ordinary Portland cement with environmental-friendly and less expensive binders such as steel slag.

The mechanical properties of CO₂ cured concrete protects may need to be evaluated to ensure the quality of products is satisfactory. The concrete units may be taken from the system for testing but this would increase the production turnover time and increase the cost.

The current disclosure explains a process in which the compressive strength of the blocks is estimated while the blocks remain on a production line. The present disclosure is directed to an automated process for evaluating the strength of concrete units and rejecting units with non satisfactory performance before they get palletized.

### Concrete Manufacturing

A method of manufacturing a concrete product as described herein may generally include mixing a composition including a binder, an aggregate, optionally an admixture, and water to produce a concrete mixture; imparting a form to the concrete mixture to provide a formed intermediate; optionally conditioning the formed intermediate to obtain a conditioned intermediate; and curing the conditioned intermediate with a gas containing carbon dioxide to obtain the concrete product.

Carbonated precast concrete is a composite material that is essentially composed of a binding medium within which are embedded fragments of aggregate. This composite material is hardened in an enriched CO₂ environment normally at its early age. Examples of carbonated precast concrete products include concrete pipes, traffic barriers, walls including retaining walls, boxes including modular boxes, culverts, tiles, pavers, foundations, slabs including hollow-core slabs, patio slabs, steps, curbs, concrete masonry units, beams, floors, columns, manholes, sewage pipes, railroad ties, and other precast concrete products.

### Aggregate

The aggregate used in carbonated precast concrete production is typically a binary blend of coarse aggregate and fine aggregate. Coarse aggregate generally refers to aggregate with particle size larger than 4.75 mm (No. 4 sieve). Fine aggregate refers to aggregate with particle size smaller than 4.75 mm. ASTM C33 specifies the quality requirements for coarse aggregate and fine aggregate. Similar specifications are also given by local government or regulatory authority, e.g., OPSS 1002, AASHTO M6 and AASHTO M80. The decision in selecting the right type and blend of aggregate is often influenced by the experience gained in manufacturing and evaluating conventional precast concrete, and also limited by supplying availability.

Among the required quality of aggregate, the maximum size and the grading of the particles are two important parameters. It is believed to affect the material cost, workability, surface quality and void content of precast concrete. Determined by the product type, application and minimum thickness (or depth) of precast concrete, the minimum clear spacing between reinforcing bars (if applicable), and the supplying availability, the maximum allowable size of coarse aggregate is often 37.5 mm (1½″). The most frequently used maximum size of coarse aggregate is 19 mm (¾″) or 9.5 mm (⅜″). For fine aggregate, it is allowed to contain a maximum of 5% (mass) particles coarser than 4.75 mm (No. 4 sieve) by ASTM C33. About the grading of aggregate, well-graded coarse or fine aggregate is generally preferred for precast concrete production, i.e., the aggregate is preferred to have relatively consistent or fair representation from every size of particle within the specified sieve sizes. For fine aggregate, an empirical factor called fineness modulus is also chosen to represent the weighted average size and distribution of the aggregate. It is obtained by summing the accumulated percentages retained on the sieves of the standard series: Nos. 4, 8, 16, 30, 50, and 100 (with openings 4.75, 2.36, 1.18, 0.6, 0.3 and 0.15 mm), and then dividing the sum by 100. The higher the fineness modulus, the coarser is the aggregate. According to the specification of ASTM C33, the fineness modulus of fine aggregate should be 2.3-3.1.

The aggregate utilized in production of CO₂ cured concrete may be normal-weight or lightweight aggregates. The aggregate may be natural or manufactured or recycled aggregates; or the combination of above.

After the aggregate suitable for manufacturing carbonated precast concrete is determined, attention is turned to other raw materials of the mixture. These raw materials include binders, water and additives (e.g., chemical admixtures and minerals).

### Binders

The binder(s) suitable for manufacturing the disclosed carbonated precast concrete should be reactive towards carbon dioxide.

The binder(s) suitable for manufacturing carbonated precast concrete may be any or a combination of cementitious and supplementary cementitious binders, which may be termed conventional “binders”. The conventional binders are the ones commonly accepted for normal (non-carbonated) precast concrete production. These binders may include: ordinary Portland cement (OPC), high alumina cement, white cement, calcium sulfoaluminate cement, magnesium cement, hydrated lime, supplementary cementitious materials including ground granulated blast furnace slag (GGBFS), fly ash, bottom ash, and natural and calcined pozzolanic materials, and OPC blended with limestone or supplementary cementitious materials.

The binder(s) suitable for manufacturing carbonated precast concrete may include emerging binders, which have weak or no hydraulic activity and also have not been recognized as supplementary cementitious materials. The main characteristics of the emerging binders are low cost and low carbon footprint, because they are either derived from waste sources or manufactured with less energy consumption and CO₂ emission than conventional cementitious binders. These binders include: belite cement, wollastonite, steel slag, stainless steel slags, bottom ash from municipal solid waste incineration, and so on.

The binder(s) suitable for manufacturing carbonated precast concrete may include any combination of conventional binders and/or emerging binders. Preferably, the binder that is suitable for manufacturing the disclosed carbonated precast concrete contains at least 10% by weight emerging binders. More preferably, the binder that is suitable for manufacturing the disclosed carbonated precast concrete contains at least 25% by weight emerging binders. More preferably, the binder that is suitable for manufacturing the disclosed carbonated precast concrete contains at least 50% by weight emerging binders. More preferably, the binder that is suitable for manufacturing the disclosed carbonated precast concrete contains at least 75% by weight emerging binders. More preferably, the binder that is suitable for manufacturing the disclosed carbonated precast concrete contains 100% by weight emerging binders.

As shown in the examples below, steel slag may be used herein as the sole component of the binder for carbonated precast concrete production. “Steel slag” herein refers to the slag by-product produced from making steel. Steel slag may include slag produced from Basic Oxygen Furnaces (BOF), also known as slag from the Linz-Donawitz (LD) process, or LD slag. Steel slag may also include slag produced from Electric Arc Furnaces (EAF). Steel slag as used herein may further include ladle slag, which is produced as a by-product from a ladle refining operation. Steel slag as used herein may further include stainless steel slag generated from stainless steel production, which is mainly generated from the argon oxygen decarburization (AOD) and/or ladle metallurgy (LM) process. In addition, steel slag may be a combination of above slags. For example, hybrid slags as used herein refers to EAF-BOF Hybrid, which is a type of steel slag formed of a mixture of EAF and BOF produced slags.

In one embodiment, the steel slag used herein has a cumulative calcium silicate content (ex: CS+C₂S+C₃S phase concentration) of at least about 15% by weight. In one embodiment, the steel slag used herein has a cumulative calcium silicate content (ex: CS+C₂S+C₃S phase concentration) of at least about 20% by weight. In one embodiment, the steel slag used herein has a cumulative calcium silicate content (ex: CS+C₂S+C₃S phase concentration) of at least about 30% by weight. In one embodiment, the steel slag used herein has a cumulative calcium silicate content (ex: CS+C₂S+C₃S phase concentration) of at least about 40% by weight. In one embodiment, the steel slag used herein has a SiO₂ content of at least about 6% or more preferably at least about 15% by weight.

The steel slag may include a mixture of coarse slag pieces and fine slag pieces. Coarse slag pieces may have a Blaine fineness less than about 50 m²/kg and fine slag pieces may have a Blaine fineness greater than about 50 m²/kg. The coarse slag pieces, the fine slag pieces, or both may be land-filled as an outcome from typical steel making process. Received steel slag originating from waste (such as land-fill and/or industrial waste) may optionally be refined. Refining the steel slag may include filtering the received steel slag to separate fine slag pieces from coarse slag pieces. Alternatively, or additionally, refining the received steel slag may also include pulverizing the steel slag to a fine powder. In some exemplary embodiments, the filtered fine pieces are pulverized while coarser pieces are not pulverized. For example, for EAF steel slag, the slag may be pulverized to a Blaine fineness of at least 50 m²/kg, and preferably about 180 m²/kg. For example, for hybrid slag steel slag (mix of EAF and BOF and ladle slag), the slag may be pulverized to a Blaine fineness of at least 100 m²/kg and preferably about 240 m²/kg. In other exemplary embodiments, the steel slag may be pulverized to a finer size. In another example, at least fifty percent of ground slag may be smaller than 100 microns, and at least ten percent of ground slag may be smaller than 50 microns, i.e., D (50)<100 microns, and D (10)<50 microns.

It will be understood that “steel slag” as used herein excludes iron slag and blast furnace slag that are typically generated during iron production and that may be used in making cement, such as pozzolanic slag.

Any potable water may be suitable for the production of the disclosed carbonated precast concrete. The addition amount of water should be controlled to the minimum value for a desired workability of concrete mixture, for the considerations of reducing conditioning time and also achieving the desired concrete density with the available manufacturing tools.

The binder may include solely steel slag and may be devoid of any other binder. It will be appreciated that expressions “binder” and “cement” have different meanings in the context of the present disclosure. A cement, such as Ordinary Portland Cement, is a kind of binder. Steel slag is another kind of binder. The binder used in the manufacturing method of the current disclosure may be devoid of cement.

In the context of the present disclosure, the expressions “cement” and “binder” have different meanings. A binder is a material that, when mixed with water will cause hardening of the mixture to obtain a concrete product. A cement is a specific type of binder. For instance, binders include Ordinary Portland Cement, steel slag, stainless steel slag, and so on. Cements include Ordinary Portland Cement, other kinds of cement. Slags are binders, but they are not considered cements. Binders may include hydraulic binders, such as cement, and non-hydraulic binders, such as slags (e.g., steel slags). A binder is referred to as “hydraulic” when reaction with water causes the hardening of the concrete product. A binder is referred to as “non-hydraulic” when another ingredient is responsible for the hardening of the concrete product. In the case of steel slag, this other ingredient is carbon dioxide. Even if the steel slag has some hydraulic properties, that is, it will harden in the presence of water, it requires carbon dioxide to achieve its full potential. Stated differently, the steel slag cured only with water will not be usable since its compressive strength will be very low. Hence, a “non-hydraulic” binder is a binder in which a fluid different than water is the main ingredient responsible for the hardening of the mixture.

### Additives

Additives that are suitable for manufacturing carbonated precast concrete include any or a combination of the following: air entraining admixture, water reducing admixture, water repellent admixture, accelerating admixture, retarding admixture, rheology modifier, efflorescence control admixture, foaming agent, alkali silica reaction inhibitor, shrinkage reducer, corrosion inhibiting admixture, pigment, mineral admixture, reinforcing fiber, polymer, and so on. The dosages of the additives used to manufacture the carbonated precast concrete may vary depending on the manufacturing process or operational parameters of carbon curing systems.

In some embodiments, the composition may include one or more chemical admixture and/or one or more mineral. The chemical admixture may include an accelerator, a retarder, a viscosity modifying agent, an air entertainer, a foaming agent, an alkali silica reaction inhibitor, an anti-wash-out, a corrosion inhibitor, a shrinkage reducer, a concrete crack reducer, a plasticizer, a super plasticizer, a sealer, a paint, a coating, a water reducer, a water repellant, an efflorescence controller, a polymer powder, a polymer latex, and a workability retainer. In at least some embodiments, the composition may be mixed with one or more of cellulose fibers, glass fibers, micro synthetic fibers, natural fibers, polypropylene fibers, polyvinyl alcohol fibers, and steel fibers.

In an application the production of a concrete mixture includes mixing water, the aggregate (such as described above), and the binder (such as described above) including steel slag.

### Mixing

In at least some embodiments, the mixing of the binder, the aggregate, and the water to produce the concrete mixture may provide a wet mixture having a mixture water-to-binder ratio. The mixing of the binder, the aggregate, and the water to produce the concrete mixture may include a dry (or dryer) mixture having a different mixture water-to-binder ratio. The binder content in the mixture may vary from 8% to 50%, depending on the binder type and/or the contemplated application of carbonated precast concrete. There are many suitable ways to perform the mixing of the concrete mixture, for example with a pan mixer.

The moisture content of the concrete mixture may be reduced from high moisture content to the optimum moisture content, and may even go below the optimum moisture content required for the carbonation reaction. In some embodiments, the mixing of the binder, the aggregate, and the water includes mixing the binder, the aggregate, and the water to obtain a water-to-binder ratio of from 0.10 to 0.5.

The presence of carbon dioxide inside the chamber/enclosed environment/vessel during the concurrent conditioning and curing process (described hereinafter) may result in a calcium carbonate precipitation that may improve strength development in concrete products.

### Forming

After a homogeneous mixture with a desired workability is obtained following the step of mixing the binder, aggregate, and water, the mixture may be emptied from the mixer and then transported to the molding place.

A molded intermediate may be obtained by imparting a form to the concrete mixture before any curing phases. Imparting a form to the concrete mixture may require an amount of the mixture to be cast into a mold with pre-set dimensions and shape, followed with being leveled. The freshly prepared concrete mixture may be transferred into the mold by any appropriate means. In some embodiments, the production method includes inserting a reinforcing material inside the mold before the casting/forming of the concrete mixture. The reinforcing material may be defined by or include bars or rods that are made at least partially, or entirely, of one or more of carbon steel, stainless steel, and fiber reinforced polymer. The reinforcing material could have other shapes, such as pellets, beads, etc.

The forming may be obtained by casting the concrete mixture in a mold to provide the formed intermediate. The concrete mixture may be formed and consolidated under compaction and/or vibration to provide the formed intermediate. Consolidation is performed to condense precast concrete mixture in the mold to the required thickness or height. Consolidation may be achieved through the known ways, such as any or a combination of vibration, compaction and compression.

If using a wet mix, it may be consolidated within the mold by internal or external vibrators. In some cases, the consolidation step lasts no more than 120 seconds. Dry cast concrete may be compacted/pressed/pressurized/formed into the mold by compaction and or vibration. The imparting of the form may include casting the concrete mixture in a shape of a precast, a concrete pipe, a box culvert, a draining product, a paving slab, a floor slab, a traffic barrier, a wall manhole, a retaining wall, a paver, a tile, or a shingle.

The mold may be made of steel, iron, aluminum, and plastic, FRP or another material. The mold may be pre-lubricated prior to casting in order to facilitate the demolding process.

### Conditioning

Once the molded intermediate is shaped, it may be demolded. In some cases, the formed intermediate may require to be maintained in the mold for a period of generally less than 24 hours. This may define a conditioning of the formed intermediate, or referred to as a pre-curing or pre-conditioning step of the formed intermediate. This process may help the formed intermediate to obtain sufficient green strength before being demolded. Such pre-curing step may happen when, for example, a wet concrete mixture is used for forming the formed intermediate. Such pre-curing, if required, may be conducted at room temperature. It may also be accelerated at elevated temperature.

The pre-conditioning of the formed intermediate may be performed until a water-to-binder ratio, which may correspond to an initial or first water-to-binder ratio after imparting the form to the formed intermediate, reaches a second water-to-binder ratio lower than the first water-to-binder ratio. After such a pre-conditioning step of the formed intermediate, the conditioned, formed, intermediate may be demolded to provide a demolded conditioned intermediate. This demolded conditioned intermediate may then go through a carbon curing step.

### Curing & Conditioning

In some embodiments, a carbon curing of the formed intermediate may be performed on the formed intermediate while the formed intermediate is still in the mold. Alternatively, the carbon curing may be performed on the demolded intermediate. More than one carbon curing step may be contemplated, such as one performed while the formed intermediate is still in the mold, and a subsequent carbon curing step on the demolded intermediate in a partially cured state. In at least some embodiments, the production method may include demolding the formed intermediate before any carbon curing.

A conditioning step may be performed so as to reduce the moisture within the consolidated precast concrete or formed intermediate (whether pre-conditioned or not). During this step, water may evaporate from the consolidated precast concrete or formed intermediate so as to reduce the moisture content thereof. The released moisture leaves numerous pores inside the consolidated precast concrete or formed intermediate, which may allow to achieve a desired CO₂ uptake and a uniform carbonation throughout the whole consolidated precast concrete or formed intermediate. In an embodiment, the conditioning step may start when the consolidated precast concrete remains in the mold. It may alternatively occur after it has been demolded. This conditioning step is optional. It may be conducted at room conditions with a temperature of 15-28° C. and a relative humidity of 20-60%. In some embodiments, the conditioning step may be assisted with a forced air circulation, such as by a blower. Other known ways of reducing the moisture, e.g. heat, may be alternatively used during the conditioning step. Alternatively, no forced air circulation may be used during the conditioning step, if energy saving is preferred and/or a longer time for conditioning is acceptable. In some embodiments, the conditioning of the formed intermediate includes conditioning the formed intermediate at a temperature ranging from 15 degrees C. to 28 degrees C. and with a relative humidity ranging from 30% to 60%. In some embodiments, the conditioning of the formed intermediate includes conditioning the formed intermediate until from 20% to 80% by weight of the water is evaporated. In some embodiments, the conditioning of the formed intermediate includes exposing the formed intermediate to a forced air flow.

The duration of the conditioning step may be influenced by multiple factors. For example, a desired moisture content or desired moisture loss from the consolidated precast concrete before carbon curing occurs. This may be affected by dimensions of the consolidated precast concrete, its geometry, its volume, and/or initial water-to-binder ratio of he consolidated precast concrete, as some possibilities. For example, in some cases, for a precast concrete with a thickness of 30 mm or greater, an initial water loss of 20-80% (by mass) may be required for a conditioned precast concrete to achieve satisfactory CO₂ uptake and strength as well as 100% CO₂ penetration, if carbonation curing is required to be completed in hours instead of days.

The degree of CO₂ penetration may be visually determined by spraying phenolphthalein indicator onto the whole cross section of carbonated precast concrete (i.e., after carbon curing), via destructive testing (e.g., compressive strength). The percentage of the area without pink color against the whole cross area is estimated as degree of CO₂ penetration. For example, a 100% CO₂ penetration is obtained if no pink color is observed in the tested cross section area, while a 50% CO₂ penetration is obtained if pink color occupies a half of the tested cross section area.

After the conditioning step, the conditioned precast concrete goes through the carbon curing step. A carbon curing is performed to obtain the concrete product. In some embodiments, the carbonation reaction between calcium-rich materials and carbon dioxide occurs once calcium leached from the material and CO₂ are dissolved in water. In a concrete sample, the carbonation reaction generally happens at a specified pore saturation. Once the pores are filled with water and the saturation rate is at or near 100%, there is little to no carbonation reaction. This observation is also valid when there is no water in the pore, or where the pore saturation is zero percent. The optimum pore saturation, or in simpler terms, the moisture content of the mix, results in the highest carbonation reaction rate. Diverging from the optimum moisture content may lead to a lower carbonation reaction and lower concrete performance.

Carbon dioxide gas, which may have a purity ranging from 5% to 99.9% may be used for carbonated precast concrete production. The pressure of carbon dioxide gas may be adjusted to from 0 MPa to 0.827 MPa (0-120 psi) during the carbonation curing process which may last from 5 minutes up to 240 hours at around 20-80 degrees C. temperature and 20-90% relative humidity.

Carbonation curing may be carried out in a sealed enclosure with CO₂ introduced either as a steady gas or as a continuously-circulated gas.

As described above, in some cases, a carbon curing step may be performed on the conditioned intermediate, either formed and still in the mold or demolded. In some embodiments, the mineralization process (i.e., the carbonation process) of the conditioned intermediate includes exposing the formed intermediate to the gas containing carbon dioxide at a pressure ranging from 0 psi to 120 psi. In some embodiments, the curing of the conditioned intermediate includes curing the conditioned intermediate for from 5 minutes to 240 hours. In some embodiments, the curing of the conditioned intermediate includes curing the conditioned intermediate at a temperature ranging from 20 degrees C. to 80 degrees C. In some embodiments, the curing of the conditioned intermediate includes curing the conditioned intermediate at a relative humidity ranging from 30% to 90%.

### Hydration

For carbonated precast concrete made of binders with hydraulic activity such as OPC, hydration curing may optionally be implemented to help carbonated precast concrete achieving full strength. During the hydration curing, carbonated precast concrete products are stored in humid environment for 1 day or longer following the general procedure known in the industry.

### Moisturizing

The carbonated precast concrete may be moisturized. This moisturizing step may include, for example, submerging the carbonated precast concrete in water; spraying the carbonated precast concrete with water; and/or misting the carbonated precast concrete with water. In some embodiments, the carbonated precast concrete is moisturized by being soaked in tap water or water saturated with hydrated lime for a period of at most 24 hours, or by being sprinkled, sprayed and/or misted with tap water. In certain embodiments, this moisturizing is performed for period of time from 0.5 to 48 hours. The preferred moisture content increase for the moisturized carbonated precast concrete is 0.5% by weight or higher, for example at least 0.55%, at least 0.6%, at least 0.65%, at least 0.7%, or at least 0.75%. There may be a delay of up to 24 hours between the proposed moisturizing step and the followed post-hardening treatment. Such a moisturizing step may be advantageous for carbonated precast concrete made of a binder with hydraulic activity. Optionally, water used for soaking/spraying may contain minerals/chemicals like efflorescence reducer admixture or water repellent. Alternatively, carbonated precast concrete may be surrounded by water vapour during a post-hardening treatment. Yet, in some embodiments, the steps of moisturizing and carbon curing may overlap or may occur concurrently.

### Concurrent Conditioning & Curing

In at least some embodiments, the step of conditioning the formed intermediate may be performed simultaneously with the step of curing. In other words, the production method may include concurrent conditioning and curing the formed intermediate. As described hereinafter, the conditioning includes drying the formed intermediate. This simultaneous conditioning and curing will be hereinafter referred to as the “SCC” process. Typically, the water-to-binder ratio is constant during the curing process because the water that is not required for the concrete composition has been removed during the conditioning process which is performed before the curing process. In the SCC process, the curing of the formed intermediate occurs while, at the same time, excess water is being evaporated out of the formed intermediate. Any precast concrete products, including but not limited to concrete masonry units, paving stones, retaining walls, slabs, traffic barriers, pipes, culverts, etc., may be produced with the SCC process.

The SCC process may be initiated once the formed intermediate is inserted in an enclosure sealed from an environment outside the enclosure. Such enclosure may be a closed chamber or vessel, in which one or more formed intermediate may be inserted. These one or more formed intermediate may be placed on racks, such as perforated racks or mesh racks allowing flow circulation along all surfaces of the formed intermediate. The CO2 curing process may be initiated immediately once the one or more formed intermediate are placed in the enclosure. As the SCC process occurs, a final water-to-binder ratio less than an initial water-to-binder ratio is obtained. In other words, while the formed intermediate is being cured, a water content of the formed intermediate decreases from an initial water-to-binder ratio to a final water-to-binder ratio. The CO2 curing process may be initiated while the formed intermediates have the initial water-to-binder ratio, i.e., water-to-binder ratio of the formed intermediate at the time the formed intermediate are placed in the enclosure, immediately once the enclosure is closed. In order to reduce the water-to-binder ratio of the formed intermediates from the initial water-to-binder ratio to a final water-to-binder ratio, i.e., the water-to-binder ratio once the CO2 curing process is terminated, the concurrent conditioning is performed. A pore saturation within the formed intermediate may thus be reduced during concurrent conditioning and carbonation curing.

Herein, the expression “concurrent” denotes that two processes occur at the same time, simultaneously. In other words, while the formed intermediate is being cured, some water is being removed out of it as part of the conditioning process. The evaporation of water/moisture from the formed intermediate during the SCC process may be obtained via forced convection within the enclosure. Forced convection may be obtain via a gas circulation device, such as a fan or blower that induced the forced convection within the enclosure. The forced convection within the enclosure may be used to control the conditioning. By increasing or decreasing the forced convection, a desired conditioning rate may be obtained and/or varied. In some embodiments, the gas velocity generated by the gas circulation device may vary from 0.1 m/s to 2 m/s. The gas velocity may be adjusted by controlling the intensity of the gas circulation device. As part of the SCC process, it may not be required to measure the gas velocity. In other words, it may not be necessary for the operator to monitor the gas velocity, as long as the intensity (e.g., rotational speed) of the gas circulation device may be controlled, including by turning it ON or OFF.

The conditioning rate may be a function of a flow circulation rate within the enclosure. Flow circulation rate may vary during the concurrent conditioning and curing. For example, in some cases, the gas circulation device may be non-operational (e.g., no inducement of flow). This implies that the carbon dioxide injected inside the enclosure may remain stationary. This may be done by varying a rotational speed of the blower.

The conditioning rate may also be a function of a temperature of the flow. For example, in some cases, the gas within the enclosure may be heated, while it is in the enclosure and/or before it being injected inside the enclosure. The SCC process may therefore include circulating heated gas about the formed intermediate in the enclosure. The gas within the enclosure may also be at room temperature or not heated by any external means. In other words, in at least some embodiments, the conditioning may be performed free of additional external sources of heat. Varying the temperature of the gas within the enclosure may be obtained via a heater. The heater may include one or more heating elements within the enclosure. In some alternatives, the body of the enclosure may be heated externally. For example, the body of the enclosure may be heated by external heating blanket. Other heating means may be contemplated, such as for example, heating wires, a heat exchanger, or solar power. A combination of one or two of the above conditioning methods may be implemented.

A temperature increase within the enclosure may increase the conditioning rate. A temperature decrease may decrease the conditioning rate. By varying the temperature within the enclosure, the conditioning rate may be controlled. The conditioning rate may thus be a function of, at least, the flow circulation rate and/or the temperature of the gas flowing within the enclosure.

The pressure inside the enclosure may also be a factor influencing the conditioning rate. In some embodiments, the SCC process may occur at a pressure higher than ambient pressure. The enclosure may be pressurized during least part of the SCC process. Pressurization of the enclosure may occur once the one or more formed intermediates are placed inside the enclosure. Pressurization may be performed immediately once the formed intermediates are placed and the enclose is sealed. The SCC process could also be initiated without pressurizing the enclosure. The enclosure may be depressurized during the SCC process or at the end thereof (i.e., once the carbonation curing reaches its desired effect on the formed intermediates). The SCC process may be performed free of additional external sources of pressure.

The conditioning rate may also be a function of a relative humidity (RH) within the enclosure, during the SCC process. An accelerated carbonation curing occurs while the relative humidity of the chamber of the enclosure is kept low. The fresh concrete products are dried or semi-dried with the help of reduced RH. Low RH may be obtained by the presence of absorbent materials and/or elevated temperature combined with air flow (e.g., with the blower) inside the enclosure for better efficiency. In embodiments where absorbent or desiccant materials, when present, may be silica gel, clay, calcium oxide, calcium chloride, molecular sieve, activated charcoal, any other industrial absorbents or a combination of any of these. The presence of the absorbent in an enclosed environment with gas circulation generated by the fan or blower) or other means) may gradually reduce the moisture content of the fresh concrete, whether the gas is heated or not. The RH inside the chamber may also be lowered using any mechanical equipment including dehumidifiers that use heating and ventilation or condensation methods for extracting water from the air.

The amount of absorbent materials required may depend on the type of material used, the total water content in the concrete products, the type of concrete products and the required or target specifications sought. The absorbent materials may be used for several cycles. The absorbent materials may be replaced by new materials after they lose their capacity for capturing moisture from the air. The absorbent materials may be place

Fresh air may be introduced into the enclosure from the environment outside of the enclosure. A port may be provided to inject air through one of the walls of the enclosure. In another embodiment, fresh air may be supplied from a sub-compartment or another source (e.g., reservoir, tank, sub-chamber) that is part of the chamber or system.

### Carbonation Curing

Aspects of the carbonation curing will now be further described.

The demolded fresh concrete may be contacted with carbon dioxide, CO₂ or a gas containing CO₂ while its moisture content is reduced during the simultaneous water extraction and CO₂ curing process. The carbon dioxide gas introduced to cure the concrete is at least 5% purity, preferably 10%, preferably 20%, preferably 30%, preferably 40%, preferably 50%, preferably 60%, preferably 70%, preferably 80%, preferably 90%, or preferably 99.5% purity. The gauge pressure of the gas will gradually increase to a range of 0.1 psi and optionally to 100 psi.

Carbon dioxide at a concentration being at least 5% by volume is injected in the enclosure. Other concentrations are contemplated, such as between 5% to 99% by volume. In embodiments where the enclosure is pressurized, the concrete products may be kept under conditioning and CO₂ pressure for a given time limit, which may be at least 10 minutes, though the simultaneous conditioning and CO₂ curing process may continue for up to 240 hours.

### System

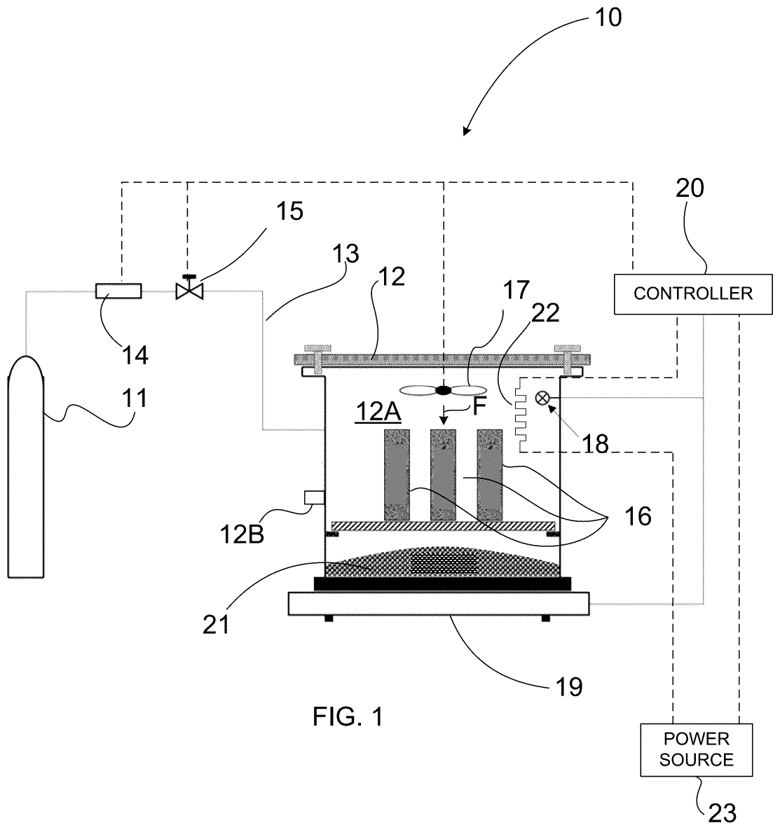

Referring now to FIG. 1, which depicts an exemplary system for conditioning and curing a concrete product is shown at 10. The system 10 includes a source of carbon dioxide 11, which may be a reservoir or tank, pneumatically connected to an enclosure 12 via a line 13. In the embodiment shown, the system 10 includes a heater 14 for heating the carbon dioxide as it flows from the source of carbon dioxide 11 to the enclosure 12. In the present configuration, the system 10 includes a valve 15 that may be selectively open or closed to allow or restrict the flow of carbon dioxide toward the enclosure 12.

The enclosure 12 defines an inner space or chamber 12A that is sized to accept the plurality of concrete products 16 to be cured. In the embodiment shown, the enclosure 12 includes top bottom and side walls interconnected to one another in an airtight manner. In the context of the present disclosure, “airtight” implies that there is little to no leakage of gas through the enclosure 12 at a pressure differential the enclosure 12 is subjected to. The pressure differential corresponds to a difference between the pressure inside the enclosure 12 and an ambient pressure outside the enclosure 12. The enclosure 12 may be structurally designed to withstand a pressure differential created by a greater pressure of the carbon dioxide inside the enclosure 12 than an atmospheric pressure outside the enclosure 12. A blower 17 may be located in the chamber 12A of the enclosure 12 and is operable to generate an airflow F that may accelerate the conditioning and/or curing process.

In some embodiments, the enclosure 12 may be used to cure the concrete products 16 using a low-pressure curing. In the context of the present disclosure, the expression “low-pressure” implies pressures that exceed the ambient pressure by at most 10% of the ambient pressure. More detail about low-pressure curing are presented in U.S. patent application Ser. No. 17/581,320 filed Jan. 21, 2022, the entire content of which is incorporated herein by reference. The enclosure 12 may be a deployable structure (e.g. bag).

The system 10 may further include one or more sensors 18, which may include one or more of a temperature sensor and a humidity sensor. The temperature sensor and humidity sensor 18 are operatively connected to the chamber 12A and are operable generate one or more signals indicative of a temperature and a humidity level inside the enclosure 12. A scale or balance 19 may support the enclosure 12 and is used to measure a weight variation of the concrete products 16 during the conditioning and curing phase. The balance 19 may send a signal indicative of a weight of the enclosure 12 containing the concrete products 16. More specifically, water content of the concrete products 16 is expected to evaporate during the conditioning and curing phase. The balance 19 may measures this weight variation and may be used to determine whether the conditioning and curing process is completed.

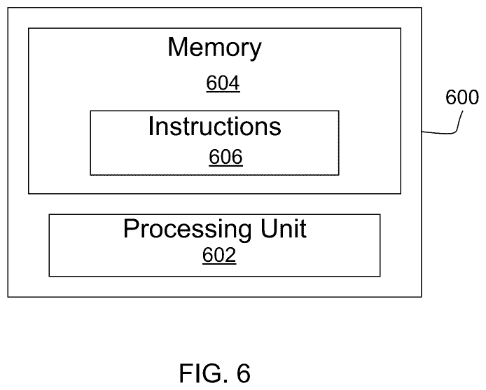

In the embodiment shown, the system 10 includes a controller 20 that may be operatively connected to the temperature and humidity sensor 18, to the balance 19, to the heater 14, to the blower 17, and to the valve 15. The controller 20 may therefore independently control the injection of carbon dioxide through the valve 15 and the actuation of the blower 17. In the embodiment shown, the controller 20 includes a computing device 600 such as the one shown and described below with reference to FIG. 6. The controller 20 may act as a data logger to save temperatures, weights, pressures, etc. data points during the conditioning and curing process. The controller 20 is operable to receive data from the temperature and humidity sensor 18 and from the balance 19; and to control operating parameters of the heater 14, the valve 15, and the blower 17. These operating parameters may include, for instance, a temperature of the heater 14, whether the valve 15 should be opened, closed, or at an intermediate position to control a flow of carbon dioxide through the valve 15, a rotational speed of the blower 17, and so on.

In the present embodiment, and as will be explained further below, the conditioning phase occurs while concrete products 16 are located inside the enclosure 12. During the conditioning phase, it is expected that water would be released from the concrete product 16. Since the enclosure 12 is closed to an environment outside the enclosure 12, it is desirable to absorb the extracted humidity from the concrete product. In the present case, a desiccant material 21 is located inside the enclosure 12 and is used to absorb excess humidity. In an alternate embodiment, the air within the enclosure may be heated to reduce its relative humidity and increase its moisture retaining capability. A combination of the desiccant material and the heating of the air may be used. A desiccant material may be a hygroscopic material that is used to induce or sustain a state of dryness in its vicinity. These desiccant materials may absorb water. The desiccant material may, in one particular example, include silica gel. Desiccant materials may be in forms other than solid, and may work through other principles, such as chemical bonding of water molecules. Desiccant materials may include, in any combinations, activated charcoal, calcium sulfate, calcium chloride, zeolites, and so on. The desiccants materials may be adsorbent materials as opposed to absorbent material. An absorbent material would contain the water by allowing the water to penetrate through it. An absorbent material may be porous and the water may be absorbed by penetrating porosities of the absorbent material. An adsorbent material will stick to water molecules. In other words, the water will be detained by the adsorbent material by being adhered to a surface of the adsorbent material. The adsorbent material may attract moistures and hold it like a magnet on its surface. It will be understood that any means able to extract humidity from the enclosure 12 during the simultaneous curing and conditioning may be used. For instance, a de-humidifier, an air conditioning, and any other suitable means may be used.

### Method of Evaluating a Performance of a Cured Concrete Product

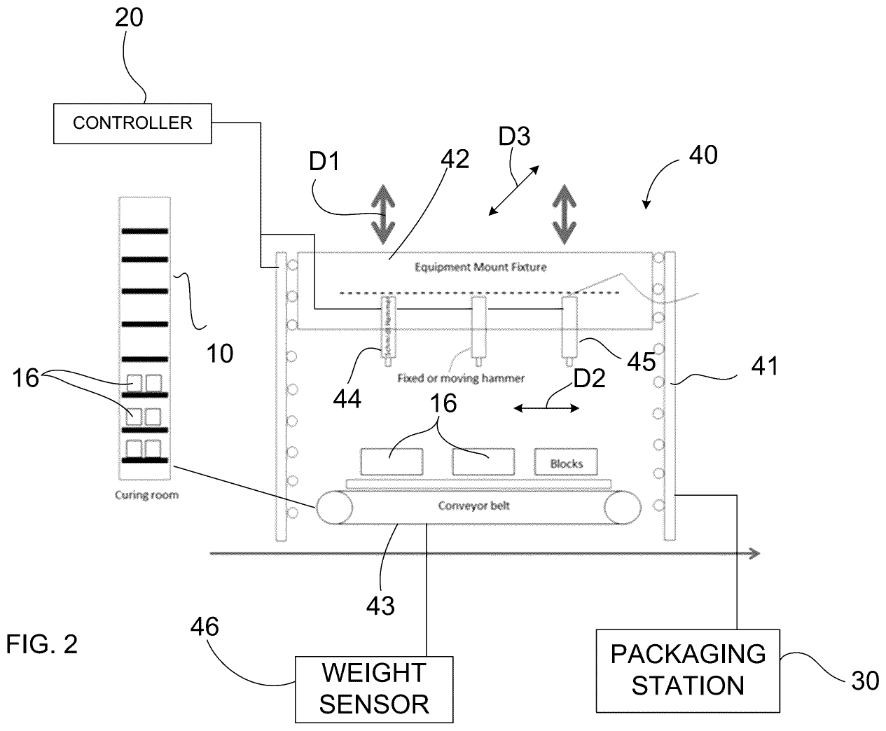

Referring to FIG. 2, as illustrated, once the concrete products 16 are cured with the system 10, they are moved towards a packaging station 30. In the packaging station 30, the concrete products 16 are palletized and prepared for shipping to customers. However, care should be taken to ensure that the concrete products 16 meet performance requirements. The performance requirements may include, for instance, compressive strength, homogeneity or uniformity, weight, and so on. In some embodiments, performance requirement may include a height of the concrete products. Thus, the concrete products 16 go through an evaluation station 40 within which equipment carries out one or more tests on the concrete products, as will be described herein.

In the current embodiment, the tests being carried on the concrete products 16 are non-destructive tests. A non-destructive test implies that, after being tested, the concrete products 16 remain substantially unchanged (e.g., they have the same size and structural strength) and are still usable by a customer. In other words, the non-destructive test does not require destruction, in whole or in part, of the concrete products 16 to evaluate its performance. The non-destructive testing does not require cutting the concrete products 16 or breaking it in any way. The concrete products 16 may undergo the non-destructive testing and, if they meet the requirements, may be shipped to a customer to be used as originally intended. The non-destructive testing does not impair a structural integrity of the concrete products being tested.

In the embodiment shown, the evaluation station 40 includes a fixture or a frame 41 for supporting the testing equipment. A carriage 42 is supported by the frame 41 and is movable relative to the frame 41 via any suitable means. For instance, the carriage 42 may be supported by the frame 41 via rails and rollers as shown. Straps and pulleys may alternatively be used. Any means allowing the carriage 42 to move relative to the frame 41 are contemplated without departing from the scope of the present disclosure. Rack and pinion gears may be used in some embodiments. The carriage 42 may move along a vertical direction D1. In some embodiments, the carriage 42 may move along a longitudinal direction D2 transverse to the vertical direction D1 and/or along a transversal direction D3 normal to both of the vertical direction D1 and the longitudinal direction D2. The longitudinal direction D2 may be parallel to a direction of movement of the concrete products 16 through the evaluation station 40.

In an alternate embodiment, the carriage 42 may be fixed and production boards supporting the concrete products 16 may be moved toward the carriage and the concrete products 16 are contacted by the testing equipment. After the estimation of the mechanical properties, the concrete products 16 are moving away from the evaluation station 40. In this example, the fixture is not moving from its position while the cured concrete products are moving. Put differently, the non-destructive testing equipment may be fixed and the concrete products may be moved towards and away from the non-destructive testing equipment.

In the disclosed embodiment, the carriage 42 is movable in at least one translational degree of freedom (DOF). The carriage 42 may displace along a plane that is generally parallel to the ground. The fixing arrangement may displace in a plurality of translational DOF in some embodiments. For example, displacement may be along a plane defined by directions D2 and D3, and/or in a transverse translational DOF, such as along the direction D1. By moving the fixing arrangement containing the non-destructive testing equipment along direction D1, the fixing arrangement may move in a vertical direction. The fixing arrangement may thus move in three translational DOF. In another embodiment, the fixing arrangement may have up to three rotational DOF. In another embodiment, the fixing arrangement may translate and rotate simultaneously. In another example, the fixing arrangement may rotate first and then translationally displace subsequently, though this could be reversed.

The carriage 42 may move until the testing equipment contacts at least one surface of cured concrete products. Some of the testing equipment are required to be pushed against the concrete products in order to get a proper measurement. Once the data is recorded and transferred to the data collection system (e.g., controller 20), the testing equipment may move to a new position to test another concrete surface and a new reading is sent to the system.

The carriage 42 may move back to its original position after all readings are taking place. The concrete products 16 meeting the requirement may be moved to the packaging station 30 and another batch of concrete products are coming into the evaluation station 40. The carriage 42 is moving to the new position to estimate the performance of the new concrete products; this evaluation cycle of cured concrete products continues.

In one embodiment, the testing equipment is fixed on the fixture. In another embodiment the testing equipment is not fixed and it is moving on the fixture. In this example, the moving testing equipment may move in one or two directions. The movement of the non-destructive testing equipment on the fixture may be controlled manually or by the controller 20. Once the testing equipment is positioned on the fixture to its desired location, the mechanical performance of the concrete units are measured. In one example, the position of all testing equipment on the fixture is adjustable; or in another words, all testing equipment are not fixed. In another example, only half of the testing equipment are fixed on the fixture and the positions of the remaining half of the testing equipment are adjustable on the fixture. In another example, the position of one of the testing equipment is adjustable on the fixture and the positions of the remaining equipment are fixed on the fixture.

The evaluation station 40 may include means for displacing the concrete products 16 therethrough. These means may include, for instance, a conveyor belt 43 operable to move the concrete products 16 along the longitudinal direction D2. Any other suitable means operable to move the concrete products 16 along the longitudinal direction D2 are contemplated.

The evaluation station 40 is equipped with non-destructive testing equipment. These may include, for instance, one or more rebound hammer 44, also referred to as a Schmidt hammer, and one or more ultrasonic pulse velocity device 45. Any suitable non-destructive equipment may be used. The rebound hammer 44 includes a mass engaged by a biasing member, such as a spring. The mass slides on a plunger within a housing. The plunger may be pressed against a surface of the concrete products 16 and the mass is accelerated with the biasing member to hit the concrete products. The mass rebounds back from the impact and a height of the mass after the rebound is measured. A concrete product with low strength and low stiffness will absorb more energy. This will cause the mass to bounce back at a lower height than if the concrete product had high strength and stiffness.

The ultrasonic pulse velocity test is used to test the quality of the concrete products by measuring a velocity of an ultrasonic pulse passing through the concrete product. Higher velocities imply a good quality and continuity of the product whereas slower velocities may indicate that the concrete product presents cracks or voids, thus, a lack of homogeneity in its structure. The ultrasonic pulse velocity device 45 may include a transmitter and a receiver. During a testing process, the transmitter and the receiver may contact one or more surfaces of one or more of the concrete products 16. Surface preparation may optionally be performed prior to having the transmitter and receiver placed on the surface(s) of the concrete products 16. Such surface preparation may provide a better contact between the pulse and the surface of the concrete unit. The quality of contact between the pulse and the surface may help to get a smoother pulse transmission to concrete. While surface preparation may be advantageous in at least some embodiments, such surface preparation is optional.

One or more weight sensor 46 may be operatively connected to the conveyor belt 43 to weigh the concrete products 16. Their weight may be an indication of whether or not the concrete products 16 are compliant with requirements. For instance, a lower weight may indicate insufficient water in the mixture. The weight sensor 46 is configured to measure a weight of the precast concrete products. Upon measuring the weight, the weight sensor 46 may generate a signal output indicative of the weight of a respective one of the precast concrete products. The weight sensor 46 may be configured to measure a combined weight of a plurality of the precast concrete products. The weight sensor 46 may include or be part of a balance. In some cases, weight measurement may involve manipulation of the products (e.g., manual measurement of the weight using a balance). In an embodiment, there is a single weight sensor. However, there may be a plurality of weight sensors. In another embodiment, the weight sensor 46 may include one or more load cells. The load cells may be installed on one equipment before the palletising station. In the depicted embodiment, the system includes a conveyor belt to transport the pre-cast concrete products between stations. In at least some embodiment, the load cells may be operatively engaged to the conveyor belt. The load cells may be configured to generate a signal output indicative of the weight of one or more precast concrete products on the conveyor belt. While in the embodiment shown the load cells are operatively engaged to the conveyor belt, the load cells may be installed on other equipment upstream of the palletising station. For instance, in a variant, the load cells are operatively engaged to the racking system so as to allow for the measurement of the pre-cast concrete products stored into the racking system.

The evaluation station 40 may be operatively connected to the controller 20, which may control its operation. For instance, the controller 20 may cause the movement of the carriage 42 and the non-destructive equipment mounted thereto along either one of the directions D1, D2, D3 until the non-destructive equipment is aligned with the concrete products to be tested. As an example, the controller 20 may cause the testing equipment to move in conjunction with the concrete products 16 such that the concrete products 16 are tested as they move on the conveyor belt 43. In some cases, the conveyor belt 43 may stop movements of the concrete products 16 during the testing. The testing equipment may include a plurality of one or more of the rebound hammer 44 and of the ultrasonic pulse velocity device 45. The testing equipment may be distributed along either or both of the longitudinal direction D2 and the transversal direction D3. For instance, the testing equipment may be installed in a matrix (e.g., 4×2) to test many products (e.g., 8) at a time. Hence, a plurality of the concrete products 16 may be tested simultaneously. The testing equipment is operatively connected to the controller 20 which gather data received from the testing equipment. The controller 20 may then identify the concrete products 16 that are not compliant with the performance requirements. These concrete products may then be removed prior to them being packaged in the packaging station 30.

The testing equipment may be simultaneously test all or a subset of the concrete products 16 exiting the curing chamber 10. Moreover, the concrete products 16 may remain positioned one relative to the other in the same way in the evaluation station 40 as they are disposed in the curing chamber. This may allow to identify discrepancy in the concrete products 16 caused by their locations in the curing chamber. This may denote a lack of uniformity of the carbon dioxide, relative humidity, temperature and so on in the curing chamber. Results from these tests may be used to adjust the curing conditions in the curing chamber 10 to at least partially mitigate these discrepancies for subsequent batches of concrete products to be cured.

A fixing arrangement, such as the carriage 42, may be used to secure the rebound hammers thereto. In an embodiment, up to six rebound hammers may be mounted on the fixing arrangement and positioned so as to impact a single one of the CO₂ cured concrete units. In an exemplary configuration, the evaluation station may includes seventy-two rebound hammers, with each subgroup of six rebound hammer positioned so as to impact a respective one of the precast concrete masonry products. In this example, up to twelve concrete products 16 could be tested concurrently and/or simultaneously in the evaluation station. Other capacity per evaluation station could be contemplated.

The plurality of rebound hammers mounted on the fixing arrangement may be equally spaced apart from each other adjacent one of the rebound hammers. A spacing between adjacent ones of the rebound hammers mounted on the fixing may be the same. A uniform distribution of rebound hammers may thus be obtained. In a particular embodiment, the spacing between adjacent ones of the rebound hammers may be 400 mm. In another particular embodiment, the spacing between adjacent ones of the rebound hammers may be 200 mm. These spacing between adjacent rebound hammers may be selected based on a size of the concrete products. For instance, the concrete products may have a size of 200 mm by 400 mm by 400 mm. The disclosed spacing may allow the rebound hammer to hit the concrete products at their centers.

In some variants, the rebound hammers may be non-uniformly distributed along one or more axis. In an example, the spacing between adjacent ones of the rebound hammers along a first axis, referred to herein as X axis (e.g., direction D2), may be 50 mm, 100 mm, 200 mm, 400 mm and 800 mm, with a different spacing along a second axis, referred to herein as Y axis (e.g., direction D3). In one example, the spacing between adjacent ones of the rebound hammers in the Y axis may be 50 mm, 100 mm, 200 mm, 400 mm and 800 mm.

The hammers are installed in a way that the height differences of concrete products do not affect the readings by the hammer. In this example, the hammer's height and position are adjusted accordingly to ensure that the concrete products are properly contacted by the hammers. The height of the hammers may be adjusted so each hammer may make an accurate reading despite the discrepancy in the concrete products' height.

In one example, one hammer may get only one reading for each production board. In another example, one hammer embedded in the fixture gets few readings from each single concrete unit. In this example, other hammers may get other readings for other units seating on the board. In another example, one hammer estimates the performance of all concrete units formed on the board. In one example, there are the same number of hammer as the number of concrete units. In this example, each hammer is dedicated to get a reading from each unit. In one example each hammer may get at least one reading from each unit. In another example, each hammer gets several readings from each unit. In another example the number of the rebound hammer may be higher than the number of CO₂ cured concrete units formed on the board.

In one embodiment, the evaluation system skips one board and takes no measurement. In this example the board moves to the packaging station 30 while the strength of no concrete unit is evaluated. In another example, the evaluation system monitors the performance of all units. In another example, the performance of 80 percent of the cured units is evaluated. In another example, the performance of 60 percent of the cured units is evaluated. In another example, the performance of half of the cured concrete units is evaluated. In another example, the performance of 40 percent of the cured units is evaluated. In another example, the performance of 20 percent of the cured units is evaluated. In another example, the performance of 20 percent of the cured units is evaluated. In another example, the performance of 10 percent of the cured units is evaluated. In another example, the performance of 5 percent of the cured units is evaluated.

In another example, the quality and performance of all products are evaluated; in another example only 1% of products are subjected to the non-destructive testing.

The data from the rebound hammer or other non destructive testing equipment are transferred to a data management system included in the controller 20. The data management system stores the data, analyzes the data and reports them. The data may be transferred to the data management system/data platform/data logger by wires or cables; in another example the generated data from the non-destructive testing equipment are transferred wirelessly with the known technologies. The data may be stored locally in the data management system; in another example the data are stored in the cloud. In another example, the data are simultaneously stored and processed locally and in the cloud. The data management system may process the data and display the performance of the concrete units, e.g. compressive strength, on the screen. The raw data and concrete performance data may be available live on the screen. The user may monitor the concrete performance of any unit tested by the testing equipment at any time any day.

The data platform may process and record the total number of failed cured concrete units per batch. The platform may compare the performance of concrete units with the minimum requirement and flags the failed units. The minimum requirement and thresholds are introduced to the system manually by a user. In another example, the data platform processes and records the total number of failed cured concrete units per day. In another example, the data platform processes and records the total number of failed cured concrete units per week. In another example, the data platform processes and records the total number of failed cured concrete units per month. In another example, the data platform processes and records the total number of failed cured concrete units per year.

The data management system may process and record the average compressive strength of concrete units per board. The platform may display the average of mechanical properties per batch. In another example, the system may calculate and display the average strength of cured concrete units per day. In another example, the system may calculate and display the average strength of cured concrete units per week. In another example, the system may calculate and display the average strength of cured concrete units per month. In another example, the system may calculate and display the average strength of cured concrete units per year.

The system identifies and flags the concrete units with non satisfying mechanical performances. For instance, the system displays an alarm on the screen if one of the tested units does not meet the minimum requirement. The alarm may be also in form of light or sound. The minimum requirement is introduced to the system and the tested units are compared with those values. In one example, the minimum compressive strength of 50 MPa is introduced to the system and the system compares the strength of the cured concrete units with this threshold. In one example, the minimum compressive strength of 40 MPa is introduced to the system and the system compares the strength of the cured concrete units with this threshold. In one example, the minimum compressive strength of 30 MPa is introduced to the system and the system compares the strength of the cured concrete units with this threshold. In one example, the minimum compressive strength of 20 MPa is introduced to the system and the system compares the strength of the cured concrete units with this threshold. In one example, the minimum compressive strength of 15 MPa is introduced to the system and the system compares the strength of the cured concrete units with this threshold. In one example, the minimum compressive strength of 10 MPa is introduced to the system and the system compares the strength of the cured concrete units with this threshold.

In another example, the disqualified concrete products may be marked and highlighted by paint or by stamp or by laser print.

The cured concrete units that do not meet the minimum requirements and flagged by the system need to be removed from the board/conveyor belt. The removal of the disqualified concrete units should be done prior to the palletization. The disqualified products are taken from the production line before they proceed to the palletization station.

The disqualified products may be removed from the production line manually. In this example, a person receives and monitors the data from the data management system, e.g. on a (touch) screen, and locates the low quality concrete units. The person then removes the disqualified unit form the line. The person may replace the disqualified concrete units with good quality concrete products before the board moves to the palletisation station.

In another embodiment, the removal of disqualified concrete units is done automatically. In this example, the disqualified concrete products are identified by cameras and sensors and they are removed by a robotic system. The same robotic system may replace the disqualified concrete units with the good quality products. In this example, the robotic system and the data management system are continuously communicating with each other.

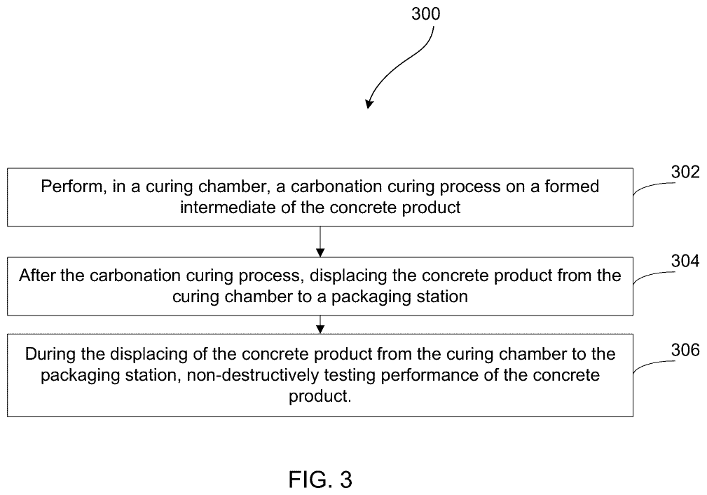

Referring now to FIG. 3, a method for manufacturing a concrete product is shown at 300 and includes performing, in a curing chamber, a carbonation curing process on a formed intermediate of the concrete product at 302; after the carbonation curing process, displacing the concrete product from the curing chamber to a packaging station at 304; and during the displacing of the concrete product from the curing chamber to the packaging station, non-destructively testing performance of the concrete product at 306.

The non-destructively testing the performance of the concrete product at 306 may include moving the concrete product through the evaluation station 40 containing non-destructive testing equipment. The non-destructive testing equipment is supported by a fixture. The non-destructive testing equipment may be movable relative to the fixture. The method includes moving the non-destructive testing equipment towards the concrete product.

The non-destructive equipment includes one or more of a rebound hammer and an ultrasonic pulse velocity device. The non-destructively testing of the performance of the concrete product at 306 may include evaluating a compressive strength of the concrete product. The evaluating of the compressive strength includes hitting or striking the concrete product with the rebound hammer 44. The concrete product may be hit more than once to obtain more than one reading. An average or other mathematical manipulation of the readings may be performed. The non-destructively testing of the performance of the concrete product may include evaluating a continuity of the concrete product. The evaluating of the continuity of the concrete product may include subjecting the concrete product to a pulse generated by an ultrasonic pulse velocity device 45.

The displacing of the concrete product at 304 may include displacing the concrete product via the conveyor belt 43. The method 300 may include weighting the concrete product while the concrete product is on the conveyor belt 43.

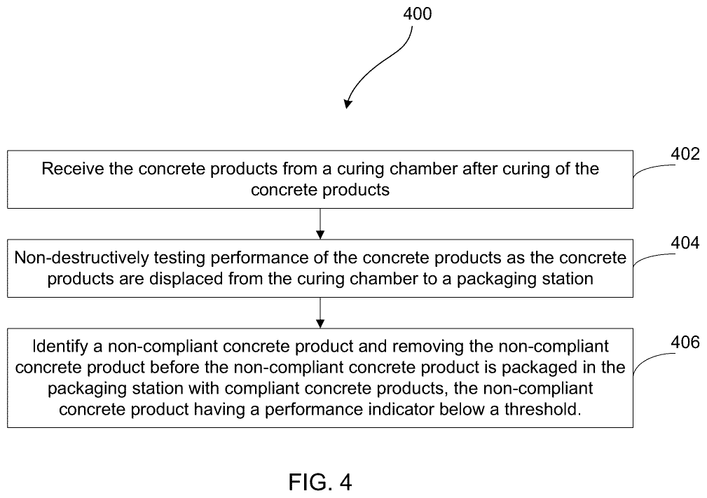

Referring now to FIG. 4, a method for detecting non-compliance with performance requirements of concrete products is shown at 400 and includes receiving the concrete products from a curing chamber after curing of the concrete products at 402; non-destructively testing performance of the concrete products as the concrete products are displaced from the curing chamber to the packaging station 30 at 404; and identifying a non-compliant concrete product and removing the non-compliant concrete product before the non-compliant concrete product is packaged in the packaging station 30 at 406. The non-compliant concrete product having a performance indicator below a threshold. The removing of the non-compliant concrete product may be carried manually by an employee or automatically by a robot.

The method 400 may include the same steps described above with reference to the method 300 of FIG. 3. Moreover, in the method 400, the non-destructively testing of the performance of the concrete products may include simultaneously testing performance of a plurality of the concrete products. In some embodiments, the simultaneously testing of the performance of the plurality of the concrete products include moving a plurality of non-destructive testing devices, such as the rebound hammer and the ultrasonic pulse velocity device relative to the plurality of the concrete products and non-destructively testing each of the plurality of the concrete products using a respective one of the non-destructing testing devices. The testing devices may be disposed in a configuration corresponding to that of the concrete products exiting the curing chamber. For instance, if the concrete products were disposed in a matrix of n by m, there may be n by m testing devices. This matrix of testing devices may be moved simultaneously such that each of the concrete products is tested at the same time. Alternatively, a subset of the concrete products may be simultaneously tested and the matrix of testing devices, which includes less testing devices than the number of concrete products to be tested, is moved relative to the concrete products until they are all tested for compliance with performance requirements.

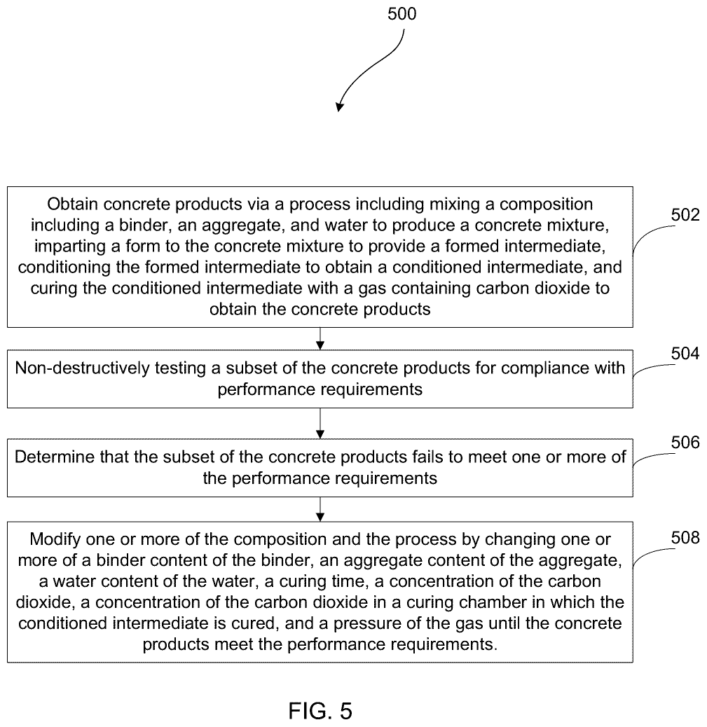

Referring now to FIG. 5, a method of manufacturing concrete products is shown at 500. The method 500 includes obtaining concrete products by: mixing a composition including a binder, an aggregate, and water to produce a concrete mixture, imparting a form to the concrete mixture to provide a formed intermediate, conditioning the formed intermediate to obtain a conditioned intermediate, and curing the conditioned intermediate with a gas containing carbon dioxide to obtain the concrete product at 502; non-destructively testing a subset of the concrete products for compliance with performance requirements at 504; determining that the subset of the concrete products fails to meet one or more of the performance requirements at 506; and modifying the composition by changing one or more of a binder content of the binder, an aggregate content of the aggregate, a water content of the water, a concentration of carbon dioxide used for the curing of the conditioned intermediate, a curing time, and a pressure of the carbon dioxide until the concrete products meet the performance requirements at 508.

It will be appreciated that not all of the concrete products 16 need to be tested. The evaluation station 40 may choose a subset of the concrete products 16 to be tested. This subset corresponds to a fraction of the concrete products 16 going through the evaluation station 40. In some cases, the concrete products 16 not selected for testing may be directed directly to the packaging station 30 by bypassing the evaluation station 40. The subset may correspond to about 1% to about 10% of the concrete products. For instance, for every 100 products passing through the evaluation station 40, 1 to 10 may be subjected to the non-destructive testing.

The precast concrete products, e.g. concrete masonry units (CMUs), are removed from the curing room or curing chamber with any known industrial equipment including a fork. The units are taken out at the end of their curing cycle. In one embodiment, the precast concrete products are stacked and stored on a racking system. The racking system may take the concrete units from the fork. The boards containing the concrete units, e.g. CMUs, are transferred from the racking system to the palletisation station using a conveyer belt. Other industrial techniques may be contemplated, such as a crane or a lift, for example.

The evaluation station 40 is located between the curing room and packaging station 30. In at least some embodiments, the evaluation station 50 may be installed immediately after the curing room or immediately before the packaging station 30.

The evaluation station 40 may further include a data logger embedded in the controller 20, a camera and a robotic arm. The controller 20 may include a data management platform, a data logger, and a screen. The robotic arm and the camera are operatively connected to the controller 20 and operable to move the concrete products 16 that are not compliant with the performance requirements out before they reach the packaging station 30.

The method 500 may include the same steps as described above for the method 300 of FIG. 3.

The modifying of the composition at 508 may include one or more of: reducing the binder content by 1% by weight if an average compressive strength of the subset of the concrete products is at least 5% higher than a minimum compressive strength requirement; reducing a curing time of the curing of the conditioned intermediate by at least 5% if the average compressive strength of subset of the concrete products is at least 5% higher than the minimum compressive strength requirement; reducing a carbon dioxide concentration of the gas containing carbon dioxide by at least 5% if the average compressive strength of subset of the concrete products is at least 5% higher than the minimum compressive strength requirement; reducing a pressure of the gas containing carbon dioxide by at least 5% if the average compressive strength of subset of the concrete products is at least 5% higher than the minimum compressive strength requirement.

In one embodiment the method 500 may be carried by the controller 20 including a data management system. The data management system may be used to optimize the concrete mix and process. The data management system may compile, process and analyze the data; the system may suggest and modify the concrete mix proportion or curing parameters based on the performance of tested concrete units. The system is then capable of optimizing the process in order to make the production faster or to reduce the cost of concrete products or combination of both. In one example, the optimization of the mix and/or process is done at the end of each production. In another example, the optimization of the mix and/or process is done at the end of each day. In another example, the optimization of the mix and/or process is done at the end of each week. In another example, the optimization of the mix and/or process is done at the end of each month. In another example, the optimization of the mix and/or process is done at the end of each year.

In one example the binder concrete, e.g. slags, in the mix is reduced by 1% if the average compressive strength of the tested units (i.e., in the subset of the concrete products) is at least 5% higher than the minimum requirement. In another example the binder concrete, e.g. slags, in the mix is reduced by 10% if the average compressive strength of the tested units is at least 5% higher than the minimum requirement. In another example the binder concrete, e.g. slags, in the mix is reduced by 20% if the average compressive strength of the tested units is at least 5% higher than the minimum requirement. In another example the binder concrete, e.g. slags, in the mix is reduced by 30% if the average compressive strength of the tested units is at least 5% higher than the minimum requirement. In another example the binder concrete, e.g. slags, in the mix is reduced by 40% if the average compressive strength of the tested units is at least 5% higher than the minimum requirement. In another example the binder concrete, e.g. slags, in the mix is reduced by at least 50% if the average compressive strength of the tested units is at least 5% higher than the minimum requirement.

In one example the CO2 curing time is reduced by 5% if the average or specified compressive strength of the tested units is at least 5% higher than the threshold. In another example the CO2 curing time is reduced by 10% if the average or specified compressive strength of the tested units is at least 5% higher than the threshold. In another example the CO2 curing time is reduced by 15% if the average or specified compressive strength of the tested units is at least 5% higher than the threshold. In another example the CO2 curing time is reduced by 20% if the average or specified compressive strength of the tested units is at least 5% higher than the threshold. In another example the CO2 curing time is reduced by 30% if the average or specified compressive strength of the tested units is at least 5% higher than the threshold. In another example the CO2 curing time is reduced by 40% if the average or specified compressive strength of the tested units is at least 5% higher than the threshold. In another example the CO2 curing time is reduced by at lest 50% if the average or specified compressive strength of the tested units is at least 5% higher than the threshold.

In one example the CO2 concentration inside the curing room is reduced by 5% if the average or specified compressive strength of the tested concrete products is at least 5% higher than the minimum requirement. In another example the CO2 concentration inside the curing room is reduced by 10% if the average or specified compressive strength of the tested concrete products is at least 5% higher than the minimum requirement. In another example the CO2 concentration inside the curing room is reduced by 20% if the average or specified compressive strength of the tested concrete products is at least 5% higher than the minimum requirement. In another example the CO2 concentration inside the curing room is reduced by 30% if the average or specified compressive strength of the tested concrete products is at least 5% higher than the minimum requirement. In another example the CO2 concentration inside the curing room is reduced by 40% if the average or specified compressive strength of the tested concrete products is at least 5% higher than the minimum requirement. In another example the CO2 concentration inside the curing room is reduced by at least 50% if the average or specified compressive strength of the tested concrete products is at least 5% higher than the minimum requirement.

In one example the CO2 pressure is reduced by 5% if the average or specified compressive strength of the tested units is at least 5% higher than the threshold. In another example the CO2 pressure is reduced by 10% if the average or specified compressive strength of the tested units is at least 5% higher than the threshold. In another example the CO2 pressure is reduced by 15% if the average or specified compressive strength of the tested units is at least 5% higher than the threshold. In another example the CO2 pressure is reduced by 20% if the average or specified compressive strength of the tested units is at least 5% higher than the threshold. In another example the CO2 pressure is reduced by 30% if the average or specified compressive strength of the tested units is at least 5% higher than the threshold. In another example the CO2 pressure is reduced by 40% if the average or specified compressive strength of the tested units is at least 5% higher than the threshold. In another example the CO2 pressure is reduced by at lest 50% if the average or specified compressive strength of the tested units is at least 5% higher than the threshold.

In the disclosed embodiment, the optimization process is executed automatically. In this case, the data management system and other equipment/system involved in the production of concrete products are connected; these systems are constantly communicating with each other and any change as the result of the optimization process is implemented automatically. In one example, the data management system sends an order to the batching plant to reduce the binder for the next batch by 10%. In another example, the evaluation system communicates with the curing room to reduce the curing time and/or the CO2 pressure and/or CO2 concentration by 10%.

### Controller

With reference to FIG. 6, an example of a computing device 600 is illustrated. For simplicity only one computing device 600 is shown but the system may include more computing devices 600 operable to exchange data. The computing devices 600 may be the same or different types of devices. The controller 20 may be implemented with one or more computing devices 600.

The computing device 600 comprises a processing unit 602 and a memory 604 which has stored therein computer-executable instructions 606. The processing unit 602 may comprise any suitable devices configured to implement the methods described herein such that the computer-executable instructions 606, when executed by the computing device 600 or other programmable apparatus, may cause the functions/acts/steps performed as part of the method described herein as described herein to be executed. The processing unit 602 may comprise, for example, any type of general-purpose microprocessor or microcontroller, a digital signal processing (DSP) processor, a central processing unit (CPU), an integrated circuit, a field programmable gate array (FPGA), a reconfigurable processor, other suitably programmed or programmable logic circuits, or any combination thereof.

The memory 604 may comprise any suitable known or other machine-readable storage medium. The memory 604 may comprise non-transitory computer readable storage medium, for example, but not limited to, an electronic, magnetic, optical, electromagnetic, infrared, or semiconductor system, apparatus, or device, or any suitable combination of the foregoing. The memory 604 may include a suitable combination of any type of computer memory that is located either internally or externally to device, for example random-access memory (RAM), read-only memory (ROM), compact disc read-only memory (CDROM), electro-optical memory, magneto-optical memory, erasable programmable read-only memory (EPROM), and electrically-erasable programmable read-only memory (EEPROM), Ferroelectric RAM (FRAM) or the like. Memory 604 may comprise any storage means (e.g., devices) suitable for retrievably storing machine-readable instructions 606 executable by processing unit 602.

The methods and systems for manufacturing concrete products described herein may be implemented in a high level procedural or object oriented programming or scripting language, or a combination thereof, to communicate with or assist in the operation of a computer system, for example the computing device 600. Alternatively, the methods and systems for manufacturing concrete products may be implemented in assembly or machine language. The language may be a compiled or interpreted language. Program code for implementing the methods and systems for manufacturing concrete products may be stored on a storage media or a device, for example a ROM, a magnetic disk, an optical disc, a flash drive, or any other suitable storage media or device. The program code may be readable by a general or special-purpose programmable computer for configuring and operating the computer when the storage media or device is read by the computer to perform the procedures described herein. Embodiments of the methods and systems for manufacturing concrete products may also be considered to be implemented by way of a non-transitory computer-readable storage medium having a computer program stored thereon. The computer program may comprise computer-readable instructions which cause a computer, or more specifically the processing unit 602 of the computing device 600, to operate in a specific and predefined manner to perform the functions described herein, for example those described in the method 400.

Computer-executable instructions may be in many forms, including program modules, executed by one or more computers or other devices. Generally, program modules include routines, programs, objects, components, data structures, etc., that perform particular tasks or implement particular abstract data types. Typically the functionality of the program modules may be combined or distributed as desired in various embodiments.

The embodiments described herein are implemented by physical computer hardware, including computing devices, servers, receivers, transmitters, processors, memory, displays, and networks. The embodiments described herein provide useful physical machines and particularly configured computer hardware arrangements. The embodiments described herein are directed to electronic machines and methods implemented by electronic machines adapted for processing and transforming electromagnetic signals which represent various types of information. The embodiments described herein pervasively and integrally relate to machines, and their uses; and the embodiments described herein have no meaning or practical applicability outside their use with computer hardware, machines, and various hardware components. Substituting the physical hardware particularly configured to implement various acts for non-physical hardware, using mental steps for example, may substantially affect the way the embodiments work. Such computer hardware limitations are clearly essential elements of the embodiments described herein, and they cannot be omitted or substituted for mental means without having a material effect on the operation and structure of the embodiments described herein. The computer hardware is essential to implement the various embodiments described herein and is not merely used to perform steps expeditiously and in an efficient manner.

The term “connected” or “coupled to” may include both direct coupling (in which two elements that are coupled to each other contact each other) and indirect coupling (in which at least one additional element is located between the two elements).

The technical solution of embodiments may be in the form of a software product. The software product may be stored in a non-volatile or non-transitory storage medium, which may be a compact disk read-only memory (CD-ROM), a USB flash disk, or a removable hard disk. The software product includes a number of instructions that enable a computer device (personal computer, server, or network device) to execute the methods provided by the embodiments.

It is noted that various connections are set forth between elements in the preceding description and in the drawings. It is noted that these connections are general and, unless specified otherwise, may be direct or indirect and that this specification is not intended to be limiting in this respect. A coupling between two or more entities may refer to a direct connection or an indirect connection. An indirect connection may incorporate one or more intervening entities. The term “connected” or “coupled to” may therefore include both direct coupling (in which two elements that are coupled to each other contact each other) and indirect coupling (in which at least one additional element is located between the two elements).

It is further noted that various method or process steps for embodiments of the present disclosure are described in the following description and drawings. The description may present the method and/or process steps as a particular sequence. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the description should not be construed as a limitation.

Furthermore, no element, component, or method step in the present disclosure is intended to be dedicated to the public regardless of whether the element, component, or method step is explicitly recited in the claims. As used herein, the terms “comprises”, “comprising”, or any other variation thereof, are intended to cover a non-exclusive inclusion, such that a process, method, article, or apparatus that comprises a list of elements does not include only those elements but may include other elements not expressly listed or inherent to such process, method, article, or apparatus.

While various aspects of the present disclosure have been disclosed, it will be apparent to those of ordinary skill in the art that many more embodiments and implementations are possible within the scope of the present disclosure. For example, the present disclosure as described herein includes several aspects and embodiments that include particular features. Although these particular features may be described individually, it is within the scope of the present disclosure that some or all of these features may be combined with any one of the aspects and remain within the scope of the present disclosure. References to “various embodiments,” “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. The use of the indefinite article “a” as used herein with reference to a particular element is intended to encompass “one or more” such elements, and similarly the use of the definite article “the” in reference to a particular element is not intended to exclude the possibility that multiple of such elements may be present.

The embodiments described in this document provide non-limiting examples of possible implementations of the present technology. Upon review of the present disclosure, a person of ordinary skill in the art will recognize that changes may be made to the embodiments described herein without departing from the scope of the present technology. Yet further modifications could be implemented by a person of ordinary skill in the art in view of the present disclosure, which modifications would be within the scope of the present technology.

1. A method for detecting non-compliance with performance requirements of concrete products, comprising:
receiving the concrete products from a curing chamber after curing of the concrete products;
as the concrete products are displaced from the curing chamber to a packaging station, non-destructively testing the concrete products for compliance with performance requirements; and
identifying a non-compliant concrete product that fails to meet one or more of the performance requirements, and removing the non-compliant concrete product before the non-compliant concrete product is packaged in the packaging station, the non-compliant concrete product having a performance indicator below a threshold.

2. The method of claim 1, wherein the non-destructively testing includes moving the concrete products through an evaluation station containing non-destructive testing equipment.

3. The method of claim 2, wherein the non-destructive testing equipment is supported by a fixture, the non-destructive testing equipment being movable relative to the fixture, the method comprising moving the non-destructive testing equipment towards the concrete product.

4. The method of claim 2, wherein the non-destructive equipment includes one or more of a rebound hammer and an ultrasonic pulse velocity device.

5. The method of claim 2, wherein the non-destructively testing includes evaluating a compressive strength of the concrete product.

6. The method of claim 1, wherein the non-destructively testing includes evaluating a compressive strength of the concrete products by striking the concrete product with a rebound hammer.

7. The method of claim 1, wherein the non-destructively testing includes evaluating a continuity of the concrete products.

8. The method of claim 7, further comprising subjecting the concrete product to a pulse generated by an ultrasonic pulse velocity device.

9. The method of claim 1, wherein the non-destructively testing includes simultaneously testing performance of a plurality of the concrete products.

10. The method of claim 9, wherein the simultaneously testing performance of the plurality of the concrete products includes moving a plurality of non-destructive testing devices relative to the plurality of the concrete products and non-destructively testing each of the plurality of the concrete products using a respective one of the non-destructing testing devices.

11. A method of manufacturing concrete products, comprising:
obtaining the concrete products via a process including:
mixing a composition including a binder, an aggregate, and water to produce a concrete mixture,
imparting a form to the concrete mixture to provide a formed intermediate,
conditioning the formed intermediate to obtain a conditioned intermediate, and
curing the conditioned intermediate with a gas containing carbon dioxide to obtain the concrete product;
non-destructively testing a subset of the concrete products for compliance with performance requirements;
determining that the subset of the concrete products fails to meet one or more of the performance requirements; and
modifying one or more of the composition and the process by changing one or more of a binder content of the binder, an aggregate content of the aggregate, a curing time, a concentration of the carbon dioxide in the gas, a concentration of the carbon dioxide in a curing chamber in which the conditioned intermediate is cured, a pressure of the gas, and a water content of the water until the concrete products meet the performance requirements.

12. The method of claim 11, wherein the non-destructively testing of the concrete products includes moving the concrete products through an evaluation station containing non-destructive testing equipment.

13. The method of claim 12, wherein the non-destructive testing equipment is supported by a fixture, the non-destructive testing equipment being movable relative to the fixture, the method comprising moving the non-destructive testing equipment towards the concrete product.

14. The method of claim 12, wherein the non-destructive equipment includes one or more of a rebound hammer and an ultrasonic pulse velocity device.

15. The method of claim 12, wherein the non-destructively testing of the concrete products includes evaluating a compressive strength of the concrete product.

16. The method of claim 15, further comprising striking the concrete product with a rebound hammer.

17. The method of claim 11, wherein the non-destructively testing of the concrete products includes evaluating a continuity of the concrete products.

18. The method of claim 17, further comprising subjecting the concrete product to a pulse generated by an ultrasonic pulse velocity device.

19. The method of claim 11, wherein the modifying of the composition includes one or more of:
reducing the binder content by 1% by weight if an average compressive strength of the subset of the concrete products is at least 5% higher than a minimum compressive strength requirement;
reducing a curing time of the curing of the conditioned intermediate by at least 5% if the average compressive strength of the subset of the concrete products is at least 5% higher than the minimum compressive strength requirement;
reducing a carbon dioxide concentration of the gas containing carbon dioxide by at least 5% if the average compressive strength of the subset of the concrete products is at least 5% higher than the minimum compressive strength requirement; and
reducing a pressure of the gas containing carbon dioxide by at least 5% if the average compressive strength of subset of the concrete products is at least 5% higher than the minimum compressive strength requirement.

20. The method of claim 11, wherein the non-destructively testing includes simultaneously testing performance of a plurality of the concrete products by moving a plurality of non-destructive testing devices relative to the plurality of the concrete products and non-destructively testing each of the plurality of the concrete products using a respective one of the non-destructing testing devices.
